(19) United States
(12) Patent Application Publication
(10) Pub. No.: US 2024/0408350 A1
(43) Pub. Date: Dec. 12, 2024

(54) Nerve Denervation Systems and Methods

(71) Applicant: Nephros Medical, Inc., Las Vegas, NV (US)

(72) Inventors: Brandon M. Repko, Mars, PA (US); Benjamin W. Bobo, Las Vegas, NV (US)

(21) Appl. No.: 18/735,937

(22) Filed: Jun. 6, 2024

Related U.S. Application Data
(60) Provisional application No. 63/471,586, filed on Jun. 7, 2023.

(51) Int. Cl. A61M 25/00; A61B 18/00; A61B 18/14

(52) U.S. Cl. CPC A61M 25/003; A61B 18/1492; A61B 2018/00166; A61B 2018/00434; A61B 2018/00511; A61M 2025/0004; A61M 2205/0216; A61M 2205/0266

(57) ABSTRACT

A renal denervation system for lysing and denervating sympathetic renal nerves that is adapted and configured to approach the renal artery extravascularly via percutaneous access. The renal denervation system includes a catheter assembly that includes a delivery catheter and at least one catheter apparatus. The catheter apparatus includes a core member that is adapted and configured to (i) transition from a pre-deployment configuration to a post-deployment curved or coiled configuration when subjected to a pre-defined temperature, wherein the core member surrounds a renal artery and, thereby, renal nerves, (ii) deliver energy to the renal artery and, thereby, renal nerves to abate activity thereof, and (iii) deliver a neurolytic agent to the renal nerves of the renal artery.

### CROSS-REFERENCES TO RELATED APPLICATIONS

The present application claims the benefit of U.S. Pat. App. No. 63/471,586, filed on Jun. 7, 2023.

### FIELD OF THE INVENTION

The present invention relates to systems and methods for treating resistant hypertension in an individual. More particularly, the present invention relates to percutaneous, extravascular systems and methods for treating resistant hypertension in an individual via renal nerve denervation.

### BACKGROUND OF THE INVENTION

Hypertension is, in many instances, the leading cause of a variety of adverse medical conditions, including myocardial infarction, heart failure, arterial aneurysms, and strokes. Unfortunately, many people are afflicted with hypertension. Indeed, the World Health Organization (WHO) estimated that in 2019 more than 1.13 billion people worldwide (approximately 15% of the world population) were afflicted with hypertension.

Hypertension; particularly, resistant (or persistent) hypertension, i.e., a blood pressure that remains above goal parameters despite the concomitant use of full doses of three (3) or more antihypertensive drugs from different classes, is often quite difficult to treat.

Hyperactivity of renal sympathetic nerves is typically associated with resistant hypertension. Various associated methods have thus been developed to abate hyperactivity of the renal sympathetic nerves.

The seminal methods to abate hyperactivity of the renal sympathetic nerves comprise denervation, i.e., abating activity, of the renal nerve.

There are currently two (2) renal denervation methods that are commonly employed to abate renal nerve activity: (i) energy-based denervation methods and (ii) chemical-based denervation methods.

Energy-based renal denervation methods comprise delivery of energy, e.g., radiofrequency (RF), heat or ultrasonic energy, into and through the renal artery wall (via percutaneous insertion of a catheter into the artery) to ablate the renal nerve plexus surrounding the renal artery. Exemplar denervation systems that are typically employed in energy-based renal denervation methods include the Symplicity®, EnligHTN®, One-Shot, Flex®, Symplicity Spyral®, Vessix®, Iberis®, TIVUS system® and Paradise® systems.

Chemical-based methods typically comprise delivery of a neurolytic agent, e.g., ethanol, directly into the adventitial and/or periadvential region surrounding the renal artery (via cannulas) to ablate the renal nerve plexus. Exemplar denervation systems that are typically employed in chemical-based renal denervation methods include the Peregrine® and Bullfrog® systems.

Although the noted renal denervation systems have generally been deemed effective means for abatement of renal sympathetic nerve activity, there are several drawbacks and disadvantages associated with both the energy-based and chemical-based renal denervation systems.

A major drawback and disadvantage associated with both energy-based and chemical-based renal denervation systems is that the use of each system typically requires general anesthetization of a patient to conduct a renal denervation procedure therewith.

A major drawback and disadvantage associated with energy-based renal denervation apparatus, such as the Symplicity, EnligHTN and One-Shot systems, is that short-term complications and long-term sequelae of applying energy, in this instance RF energy, from the inner lining (intima) of the renal artery to the outer wall of the artery are not well defined. Indeed, RF energy applied from within the renal artery can, and often will, result in transmural renal artery injury, which can lead to late stenosis, thrombosis, renal artery spasm, embolization of debris into the renal parenchyma, and/or other complications associated with RF-associated injury to the renal artery.

A further major drawback and disadvantage associated with energy-based renal denervation systems is that there is often uneven or incomplete ablation of the renal nerve plexus; particularly, if there are existing anatomic anomalies, or atherosclerotic or fibrotic disease in the intima of the renal artery, resulting in non-homogeneous and uneven delivery of RF energy to the renal artery and renal nerve plexus. To compensate for patient-to-patient physiological variations in renal artery structure, operators of energy-based renal denervation apparatus often use excess levels of RF energy to ablate the renal nerve plexus that runs along the adventitial plane of the renal artery, which can, and often will exacerbate transmural renal artery injury induced by the RF energy.

A major drawback and disadvantage associated with chemical-based renal denervation systems, such as the Peregrine® and Bullfrog® systems, which, as indicated above, employ one or more cannulas to deliver a neurolytic agent into the adventitial and/or periadvential region surrounding the renal artery, typically require multiple applications of the neurolytic agent to achieve the desired effect, i.e., abatement of sympathetic renal nerve activity. It is also often difficult to deliver the appropriate volume of the neurolytic agent into the adventitial and/or periadvential region of the renal artery with the cannula(s).

A further drawback and disadvantage associated with chemical-based renal denervation systems is that such systems are typically devoid of means for precise, controlled, and adjustable depth of delivery of a neurolytic agent. Indeed, most chemical-based renal denervation systems do not have any physical constraints regarding the length of the cannula that is employed, and/or injection depth.

A further drawback and disadvantage associated with chemical-based renal denervation systems is that such systems often employ intravascular catheter systems that are too large to access associated renal artery structures, such as the segmental renal arteries disposed at the distal end of a main renal artery and, thus, fail to ablate the renal nerves disposed on the extravascular surfaces of those associated renal artery structures.

A further major drawback and disadvantage associated with chemical-based renal denervation systems is that the cannula(s), which are employed to deliver the neurolytic agent, must penetrate into the renal artery to do so, which can result in extravascular bleeding, hematoma formation, renal arterial dissection, renal artery stenosis, nerve injury, hypotension, and post-operative pain associated therewith.

There is thus a need for improved renal denervation systems and associated methods; particularly, percutaneous, extravascular renal denervation systems and methods, which effectively abate sympathetic renal nerve activity without damaging the renal artery and its associated structures.

It is thus an object of the present invention to provide improved renal denervation systems and associated methods; particularly, percutaneous, extravascular renal denervation systems and methods, which effectively abate sympathetic renal nerve activity without damaging the renal artery and its associated structures.

It is another object of the present invention to provide improved renal denervation systems and associated methods; particularly, percutaneous, extravascular renal denervation systems and methods, which abate sympathetic renal nerve activity percutaneously from outside of the renal artery lumen, i.e., extravascularly.

It is another object of the present invention to provide improved renal denervation systems and associated methods; particularly, percutaneous, extravascular renal denervation systems and methods, which provide precise and consistent delivery of pharmacological agents; particularly, neurolytic agents proximate the renal nerve plexus to abate sympathetic renal nerve activity.

It is another object of the present invention to provide improved renal denervation systems and associated methods; particularly, percutaneous, extravascular renal denervation systems and methods, which are adapted to deliver precise and controlled (i) energy, e.g., radiofrequency (RF), heat or ultrasonic energy to renal nerves, and (ii) pharmacological agents; particularly, neurolytic agents proximate the renal nerve plexus to abate sympathetic renal nerve activity.

It is another object of the present invention to provide improved renal denervation systems and associated methods; particularly, percutaneous, extravascular renal denervation systems and methods, which are adapted to access and ablate renal nerves disposed on the extravascular surface of associated renal artery structures.

It is another object of the present invention to provide improved renal denervation systems and associated methods; particularly, percutaneous, extravascular renal denervation systems and methods, which do not require general anesthesia and, hence, can be performed with local anesthesia or moderate conscious sedation of a patient.

### SUMMARY OF THE INVENTION

The present invention is directed to renal denervation systems, apparatus and methods for percutaneous, extravascular abatement of renal nerve activity.

The renal denervation systems generally comprise a catheter assembly and a catheter control system.

In a preferred embodiment, the catheter assembly comprises a delivery catheter and a catheter apparatus adapted to receive and deliver energy and a pharmacological agent proximate to a renal artery and, thereby, renal nerve.

In a preferred embodiment, the catheter control system comprises processing means, an energy delivery module, agent delivery module, imaging module and power supply means.

As discussed herein, in a preferred embodiment, the energy delivery module is adapted to generate and provide energy to the catheter apparatus to position and configure the catheter apparatus and, in some embodiments, abate hyperactivity of the renal nerve.

In a preferred embodiment, the agent delivery module is adapted and configured to modulate the delivery of a pharmacological agent, such as a neurolytic agent, proximate to the renal nerve via a catheter assembly.

In a preferred embodiment, the imaging module is adapted and configured to receive and process visual images captured via an endoscope.

As also discussed herein, the renal denervation systems and apparatus (and methods associated therewith) denervate the renal nerves from outside of the renal artery, i.e., extravascularly, where it is more effective and much safer.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1A is a schematic illustration of human renal anatomy;

FIG. 1B is a further schematic illustration of human renal anatomy showing a portion of a renal artery and the renal nerves, i.c., a renal nerve plexus;

FIG. 1C is a cross-sectional view taken along the radial plane A-A of FIG. 1B;

FIG. 2 is a schematic illustration of one embodiment of a renal denervation system, in accordance with the invention;

FIG. 3 is a perspective view of one embodiment of a delivery catheter, in accordance with the invention;

FIG. 4 is a partial perspective view of one embodiment of a catheter apparatus, in accordance with the invention;

FIG. 5A is a partial perspective view of another embodiment of a catheter apparatus, in accordance with the invention;

FIG. 5B is a partial perspective view of one embodiment of a catheter assembly comprising the catheter apparatus illustrated in FIG. 5A, in accordance with the invention;

FIGS. 5C and 5D are partial perspective views of one embodiment of a catheter assembly comprising the catheter apparatus illustrated in FIG. 4, in accordance with the invention;

FIG. 6 is a partial perspective view of the catheter apparatus illustrated in FIG. 5A partially surrounding a renal artery, in accordance with the invention;

FIG. 7 is an illustration of the catheter apparatus shown in FIG. 4 surrounding a renal nerve plexus region, in accordance with the invention;

FIG. 8 is an illustration of the catheter apparatus shown in FIG. 4 similarly surrounding a renal nerve plexus region, in accordance with the invention; and

FIG. 9 is an illustration of one embodiment of a hand grip actuator in accordance with the invention.

### DETAILED DESCRIPTION OF THE PREFERRED EMBODIMENT

Before describing the present invention in detail, it is to be understood that this invention is not limited to particularly exemplified apparatus, systems, materials, compositions, structures or methods as such may, of course, vary. Thus, although a number of apparatus, systems, materials, compositions, structures and methods similar or equivalent to those described herein can be used in the practice of the present invention, the preferred apparatus, systems, materials, compositions, structures and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

As used in this specification and the appended claims, the singular forms “a”, “an” and “the” include plural referents unless the content clearly dictates otherwise.

### Definitions

The terms “superelastic” and “pseudoclastic” are used interchangeably herein, and mean and include a shape-memory alloy (SMA) that has the inherent capability to undergo deformation at one temperature, stay in the deformed configuration when the force(s) exerted to deform the SMA has been removed, then recover the original deformed configuration upon heating the SMA above a transformation temperature.

The terms “superelastic” and “pseudoelastic” thus mean and include shape-memory Ni—Ti alloys, such as Nitinol® alloys.

The terms “extravascular” and “perivascular” as used in connection with the renal denervation systems of the invention, mean situated or occurring outside the vascular system, e.g., outside of the internal lumen of a patient's renal arteries.

The terms “pharmacological agent”, “active agent” and “pharmacological formulation” are used interchangeably herein, and mean and include an agent, drug, compound, composition of matter or mixture thereof, including its formulation, which provides some therapeutic, often beneficial, effect. This includes any physiologically or pharmacologically active substance that produces a localized or systemic effect or effects in animals, including warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The terms “pharmacological agent”, “active agent” and “pharmacological formulation” thus mean and include, without limitation, antibiotics, anti-arrhythmic agents, anti-viral agents, analgesics, anti-inflammatory agents, anti-ncoplastics, anti-spasmodics, modulators of cell-extracellular matrix interactions, proteins, hormones, growth factors, matrix metalloproteinases (MMPs), enzymes and enzyme inhibitors, anticoagulants and/or antithrombotic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

The terms “pharmacological agent”, “active agent” and “pharmacological formulation” thus include, without, limitation the following neurolytic agents: ethanol, phenol, glycerol, local anesthetics in relatively high concentration (e.g., lidocaine, or other agents such as bupivicaine, tetracaine, benzocaine, etc.); anti-arrhythmic drugs with neurotoxic properties (e.g., amiodarone and flecainide), botulinum toxin, digoxin, cardiac glycosides, guanethidine; and heated fluids including heated saline, hypertonic saline, hypotonic fluids and potassium chloride; and cooled fluids, such as liquid nitrogen.

The terms “neurolytic agent” and “neurolytic” are used interchangeably herein, and mean and include an agent, drug, compound, composition of matter or mixture thereof, including its formulation, which are adapted to neutralize or induce neutralization of nerves and nerve structures, i.e., denervation.

The term “neutralize” as used herein in connection with a renal denervation system of the invention means and includes the process of rendering a renal nerve at least partially ineffective and/or unable to conduct motor and sensory signals.

The terms “pharmacological agent”, “active agent” and “pharmacological formulation” thus include, without, limitation the following anti-inflammatory agents: alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, triamcinolone, betamethasone, clopirac, cloticasone propionate, cormethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, momiflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, and zomepirac sodium.

The terms “pharmacological agent”, “active agent” and “pharmacological formulation” also mean and include, without limitation, the following antibiotics: aminoglycosides, cephalosporins, chloramphenicol, clindamycin, erythromycins, fluoroquinolones, macrolides, azolides, metronidazole, penicillins, tetracyclines, trimethoprim-sulfamethoxazole, gentamicin, and vancomycin.

The term “polymer”, as used herein thus means and includes, without limitation, the following non-biodegradable polymers: polytetrafluoroethylene (PTFE), ethylene tetrafluoroethylene (ETFE), fluorinated ethylene propylene (FEP), polyoxymethylene (DELRIN®), polyether block ester, polyurethane (Polyurethane 85A), polypropylene (PP), polyvinylchloride (PVC), polyether-ester (ARNITEL®), ether or ester based copolymers (butylene/poly (alkylene ether) phthalate and/or other polyester elastomers such as HYTREL®), polyamide (DURETHAN® or CRISTAMID®), elastomeric polyamides, block polyamide/ethers, polyether block amide (PEBAX®), ethylene vinyl acetate copolymers (EVA), silicones, polyethylene (PE), MARLEX® high-density polyethylene, MARLEX® low-density polyethylene, linear low density polyethylene (REXELL®), polyester, polybutylene terephthalate (PBT), polyethylene terephthalate (PET or Dacron®), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (KEVLAR®), polysulfone, nylon, nylon-12 (GRILAMID®), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (SIBS and/or SIBS 50A), polycarbonates, or the like.

The terms “prevent” and “preventing” are used interchangeably herein, and mean and include reducing the frequency or severity of a disease or condition. The term does not require an absolute preclusion of the disease or condition. Rather, this term includes decreasing the chance for disease occurrence.

The terms “treat” and “treatment” are used interchangeably herein, and mean and include medical management of a patient with the intent to cure, ameliorate, stabilize, or prevent a disease, pathological condition, or disorder. The terms “treat” and “treatment” include “active treatment”, i.e., treatment directed specifically toward the improvement of a disease, pathological condition, or disorder, and “causal treatment”, i.e., treatment directed toward removal of the cause of the associated disease, pathological condition, or disorder.

The terms “treat” and “treatment” further include “palliative treatment”, i.e., treatment designed for the relief of symptoms rather than the curing of the disease, pathological condition, or disorder, “preventative treatment”, i.e., treatment directed to minimizing or partially or completely inhibiting the development of the associated disease, pathological condition, or disorder, and “supportive treatment”, i.e., treatment employed to supplement another specific therapy directed toward the improvement of the associated disease, pathological condition, or disorder.

The term “comprise” and variations of the term, such as “comprising” and “comprises,” means “including, but not limited to” and is not intended to exclude, for example, other additives, components, integers or steps.

The term “proximate”, as used herein in connection with a renal denervation system of the invention means and includes placement of the renal denervation system and/or a component thereof, such as a delivery catheter or catheter apparatus thereof, in close proximity to a renal artery and/or associated renal artery structures, including, without limitation, the main renal arteries (including proximal, medial and distal sections of the main renal arteries); the segmental arteries, such as the superior segmental renal artery, inferior segmental renal artery, posterior segmental artery, inferoanterior segmental renal artery, and anterosuperior segmental renal artery; the accessory renal arteries, such as the upper pole accessory arteries and lower pole accessory arteries; the suprarenal arteries, such as the inferior suprarenal arteries and middle suprarenal arteries; and the capsular branches of main renal arteries.

The present invention is directed to renal denervation systems, apparatus and methods for percutaneous and extravascular abatement of renal nerve activity.

Although the renal denervation systems, apparatus and methods of the invention are described primarily in connection with denervation of the renal nerves, it is understood that the renal denervation systems, apparatus and methods are not limited to solely denervating renal nerves. As one having ordinary skill in the art will readily appreciate, the renal denervation systems, apparatus and methods of the invention can also be readily employed to denervate other nerve structures, such as, by way of example, facet nerves, hepatic artery nerves, splenic artery nerves, pulmonary artery nerves, and associated nerve structures.

As one having ordinary skill in the art will also readily appreciate, in addition to treating hypertension, the renal denervation systems, apparatus and methods of the invention can also be readily employed to treat other diseases and disorders, such as, without limitation, insulin resistance, genetic metabolic syndromes, ventricular tachycardia, atrial fibrillation, arrhythmia, inflammatory diseases, obesity, hyperglycemia, hyperlipidemia, and endocrine diseases.

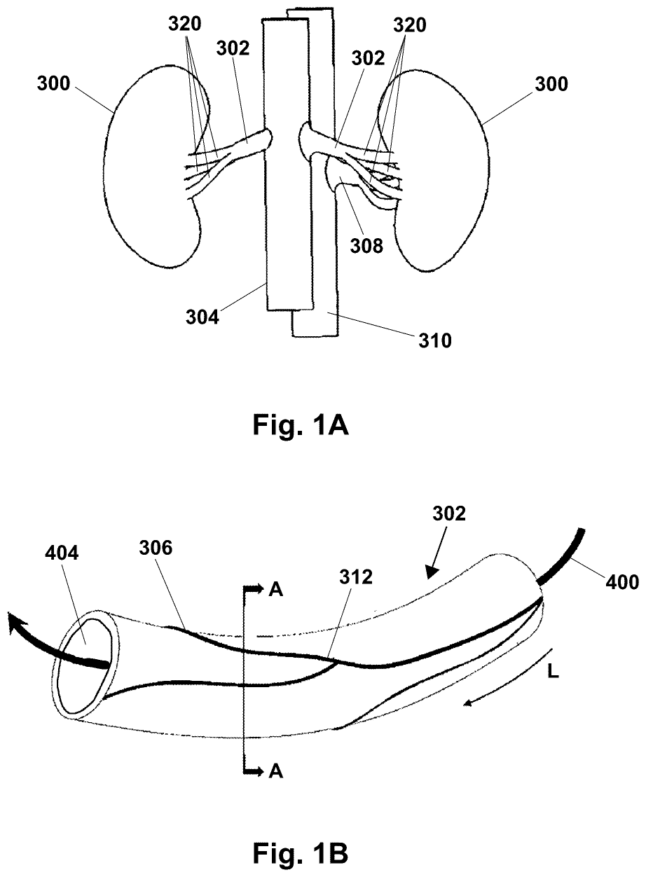

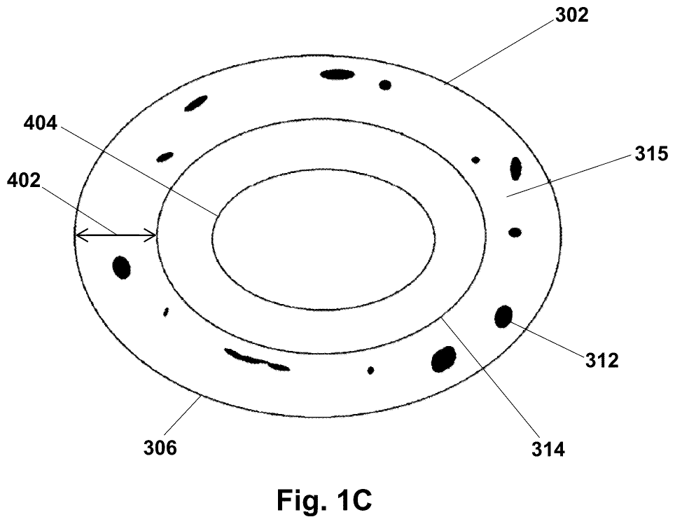

Referring first to FIG. 1A, there is shown an illustration of human renal anatomy. As illustrated, the kidneys 300 are supplied with oxygenated blood by renal arteries 302 and associated renal artery structures 320 (in this instance, segmental renal arteries), which are connected to the heart by the abdominal aorta 304. Deoxygenated blood flows from the kidneys 300 to the heart via renal veins 308 and the inferior vena cava 310.

Referring now to FIGS. 1B and 1C, there are shown illustrations of a portion of a renal artery 302 and renal nerves 312 (referred to herein as a “renal nerve plexus” and denoted “306”), where FIG. 1C illustrates a cross-sectional view taken along the radial plane A-A of FIG. 1B. As illustrated in FIG. 1B, the renal artery 302 has a lumen 404 through which the blood 400 flows.

As further illustrated in FIGS. 1A and 1B, the renal nerves 312 are located in proximity to the adventitia (denoted “402” in FIG. 1C) of the renal artery 302 and run along the renal artery 302 in a lengthwise direction (denoted by arrow “L”). More specifically, renal nerves 312 are situated in circumferential tissue 315 surrounding the outer wall of the renal artery 302. The circumferential tissue 315 can, and many times does, include other tissue, such as lymphatics and capillaries.

In the conventional denervation methods based on applying denervation energy to abate activity of the renal nerves 312, a catheter is inserted into the renal artery lumen 404 and delivers energy, e.g., heat energy, to denervate the target renal nerves 312. As indicated above, during this process, the denervation energy can damage the adventitia 402 of renal artery 302 before it reaches the renal nerves 312. Further, a portion of the denervation energy is often absorbed by the adventitia 402 of the renal artery 302, which can, and often does, reduce the effectiveness of the denervation energy.

Accordingly, as discussed in detail herein, the renal denervation systems, apparatus and methods of the invention denervate the renal nerves 312 from outside of the renal artery 302, i.e., extravascularly, which is more effective and much safer.

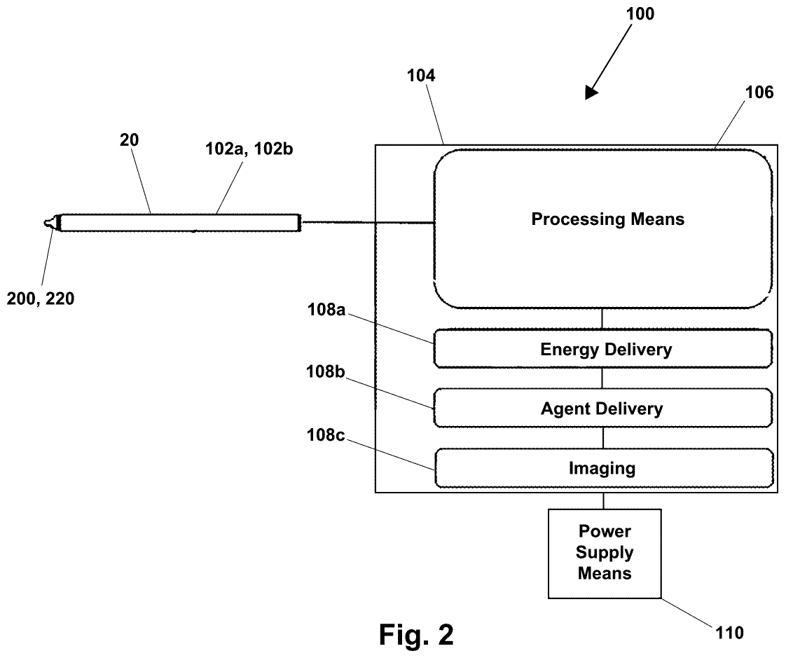

Referring now to FIG. 2, there is shown a schematic block diagram of a renal denervation system of the invention (denoted “100”). As illustrated in FIG. 2, the renal denervation system 100 generally comprises a catheter assembly (denoted “102a” and “102b” herein) and a catheter control system 104.

### Renal Denervation Systems

As further illustrated in FIG. 2, the catheter control system 104 comprises processing means 106, and an energy delivery module 108a, agent delivery module 108b, imaging module 108c and power supply means 110, which are in communication with the processing means 106.

As discussed in detail below, in a preferred embodiment, the energy delivery module 108a is adapted to generate and provide energy to the catheter apparatus of the invention, e.g., catheter apparatus 200, 220, to position and configure the catheter apparatus and, in some embodiments, abate activity of renal nerves, the agent delivery module 108b is adapted and configured to modulate the delivery of a pharmacological agent, such as a neurolytic agent, proximate the renal nerve via a catheter assembly, e.g., catheter assemblies 102a, 102b, the imaging module 108c is adapted and configured to receive and process visual images captured via an endoscope, and the power supply means 110 is adapted to supply electrical power to the catheter control system 104.

According to the invention, the energy generated and provided by the energy module 108a can comprise, without limitation, radiofrequency (RF) energy, electrical energy, laser energy, ultrasonic energy and high-intensity focused ultrasound (HIFU) energy.

As also discussed in detail herein, the catheter assemblies of the invention comprise a delivery catheter and a catheter apparatus.

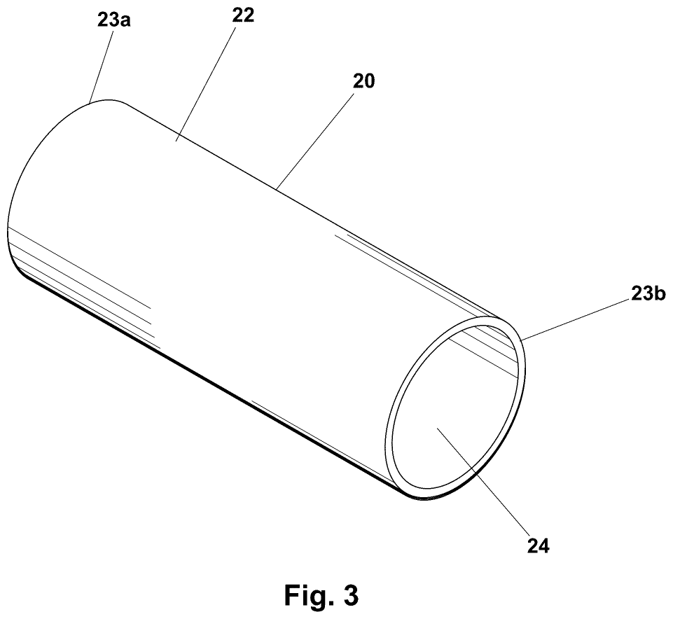

Referring now to FIG. 3, there is shown one embodiment of a delivery catheter of the invention (denoted “20”). As illustrated in FIG. 3, the delivery catheter comprises a delivery catheter sheath 22 comprising a proximal end 23a, a distal end 23b, and an internal lumen 24.

According to the invention, the delivery catheter sheath 22 can comprise various biocompatible polymers, including, without limitation, one of the aforementioned biocompatible polymers.

In a preferred embodiment, the delivery catheter sheath 22 comprises polyurethane (PU).

According to the invention, delivery catheter sheath 22 can comprise various lengths to accommodate access to a renal nerve plexus and, hence, renal nerves thereof.

In a preferred embodiment, the delivery catheter sheath 22 comprises a length in the range of approximately 1.0 cm to approximately 100.0 cm, more preferably, a length in the range of approximately 5.0 cm to approximately 50.0 cm.

According to the invention, the internal lumen 24 of the delivery catheter sheath 22 can further comprise various diameters.

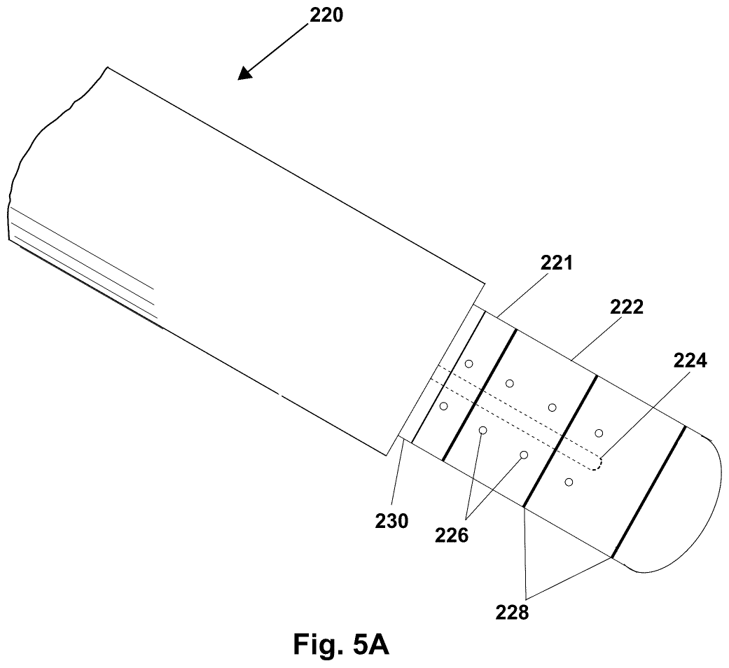

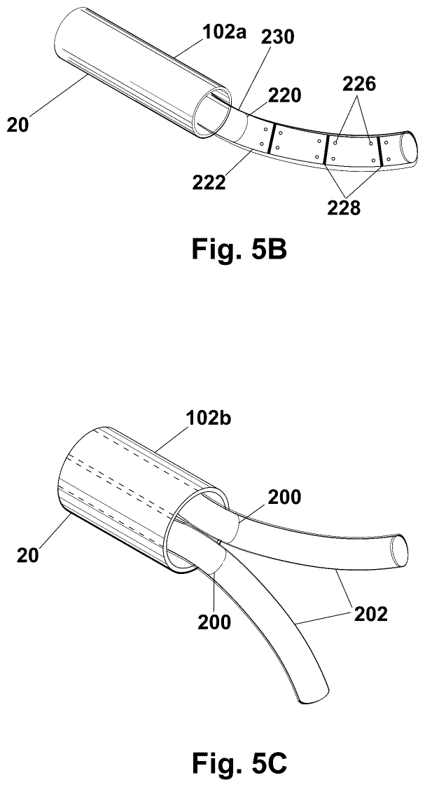

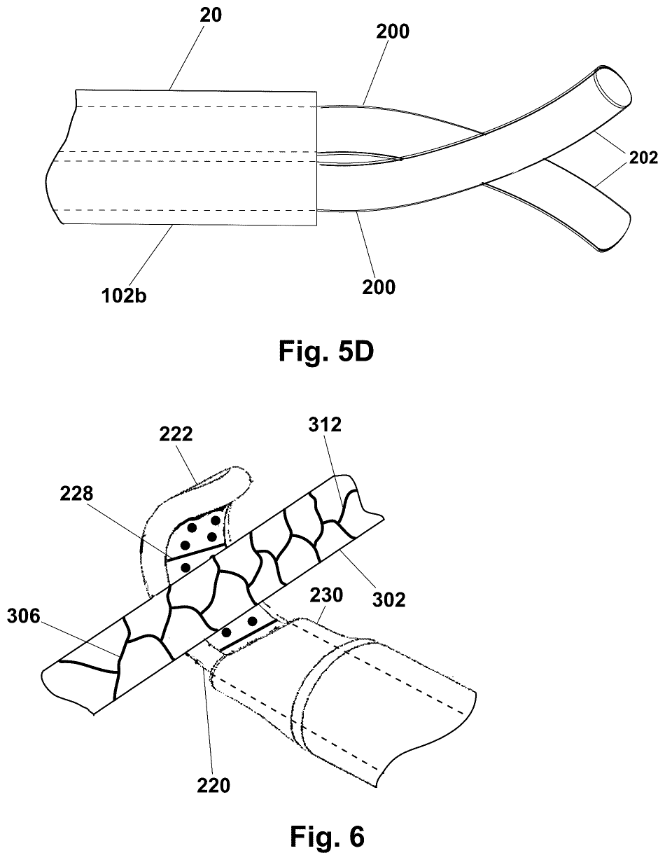

In a preferred embodiment, the internal lumen 24 is sized and configured to receive at least one catheter apparatus, e.g., catheter apparatus 200, 220, more preferably, between two (2) to four (4) catheter apparatus, as illustrated in FIGS. 5C and 5D showing catheter assembly 102b.

In some embodiments, the internal lumen 24 is also sized and configured to accommodate placement of an endoscope apparatus therein to provide an operator with visual guidance during a renal denervation procedure.

In some embodiments of the invention, the internal lumen 24 of the delivery catheter sheath 22 thus comprises an inner diameter in the range of approximately 10.0 um to approximately 25.0 mm, more preferably, an inner diameter in the range of approximately 2.0 mm to approximately 20.0 mm.

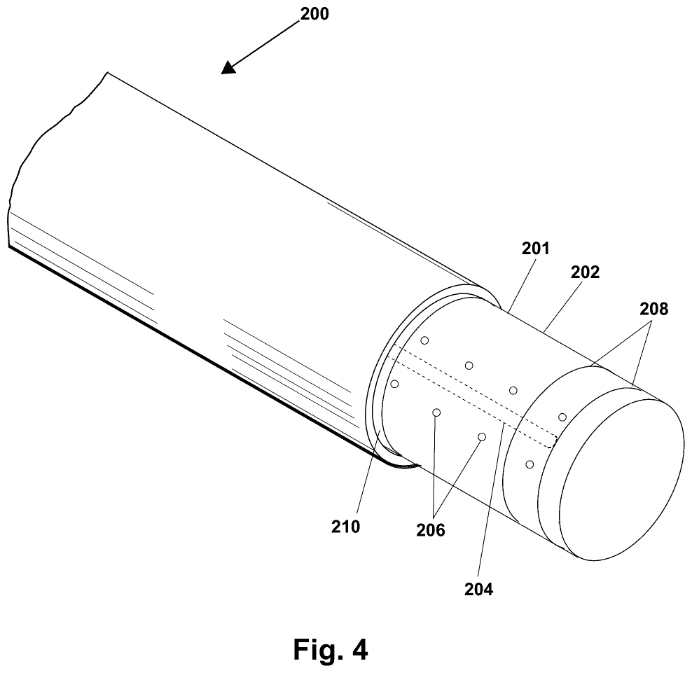

Referring now to FIG. 4, there is shown one embodiment of a catheter apparatus of the invention (denoted “200”). As illustrated in FIG. 4, in a preferred embodiment, the catheter apparatus 200 comprises a core member 202 and an outer sheath 210, which is disposed proximate the outer surface 201 of the core member 202.

According to the invention, the outer sheath 210 can similarly comprise various biocompatible polymers, including, without limitation, one of the aforementioned biocompatible polymers.

According to the invention, the outer sheath 210 can also comprise a polymer composition outer coating comprising one of the aforementioned biocompatible polymers. In some embodiments, the outer sheath 210 comprises at least one radiopaque marker.

In a preferred embodiment of the invention, the core member 202 comprises an elongated structure that is particularly well-suited for accessing and conforming to substantially tubular anatomical structures, such as a renal artery and/or its associated renal artery structures and providing physiologically acceptable radial or coil strength and longitudinal flexibility.

According to the invention, the core member 202 can comprise various cross-sectional shapes, such as the substantially circular cross-sectional shape illustrated in FIG. 4 and the planar, substantially rectangular cross-sectional shape illustrated in FIGS. 5A and 6.

Referring back to FIG. 4, in a preferred embodiment, the core member 202 further comprises an internal lumen 204 that extends therethrough and a plurality of fenestrations 206 that are in communication with the internal lumen 204.

In a preferred embodiment, the internal lumen 204 is sized and configured to receive a pharmacological agent (or formulation thereof) therein and the fenestrations 206 are sized and configured to allow a pharmacological agent delivered into and through the internal lumen 204 to be perfused through the fenestrations 206 and, hence, delivered to a renal nerve plexus or structure when disposed proximate thereto.

According to the invention, the fenestrations 206 can comprise any suitable size and shape. The core member 202 can also comprise any quantity of fenestrations 206, which can be disposed on any region of the core member 202 in any suitable pattern, such as the pattern shown in FIG. 4.

As further illustrated in FIG. 4, in a preferred embodiment, the core member 202 comprises a plurality of radiopaque makers 208. According to the invention, the core member 202 can comprise any quantity of radiopaque makers 208 disposed in any suitable pattern along the length of the core member 202.

Referring now to FIG. 5A, there is shown another embodiment of a catheter apparatus of the invention (denoted “220”).

As illustrated in FIG. 5A, in a preferred embodiment, the catheter apparatus 220 similarly comprises a core member 222 and an outer sheath 230, which is disposed proximate the outer surface 221 of the core member 222.

According to the invention, the outer sheath 230 can similarly comprise various biocompatible polymers, including, without limitation, one of the aforementioned polymers.

The outer sheath 230 can also similarly comprise a polymer composition outer coating comprising one of the aforementioned biocompatible polymers.

In some embodiments, the outer sheath 230 similarly comprises at least one radiopaque marker.

As further illustrated in FIG. 5A, the core member 222 comprises a planar substantially rectangular cross-sectional shape, which is similarly particularly suited for accessing and conforming to substantially tubular anatomical structures, such as a renal artery and/or its associated renal artery structures.

In a preferred embodiment, the core member 222 similarly comprises an internal lumen 224 that extends therethrough and a plurality of fenestrations 226 that are in communication with the internal lumen 224.

In a preferred embodiment, the internal lumen 224 is similarly sized and configured to receive a pharmacological agent (or formulation thereof) therein and the fenestrations 226 are similarly sized and configured to allow a pharmacological agent delivered into and through the internal lumen 224 to be perfused through the fenestrations 226 and, hence, delivered to a renal nerve structure when disposed proximate thereto.

As further illustrated in FIG. 5A, in a preferred embodiment, the core member 222 similarly comprises a plurality of radiopaque makers 228. According to the invention, the core member 222 can also comprise any quantity of radiopaque makers 228 disposed in any suitable pattern along the length of the core member 222.

According to the invention, the core members 202, 222 can comprise various biocompatible materials, including polymers, stainless steels and alloys comprising same, magnesium and alloys comprising same, and shape memory alloys, including, without limitation, nickel-titanium (Ni—Ti) alloys.

According to the invention, the core members 202, 222 can thus comprise a shape memory polymer (SMP), such as a segmented thermoplastic polyurethane (STPU).

In a preferred embodiment, the core members 202, 222 comprise a Ni—Ti alloy, more preferably, as discussed in detail below, a shape-memory Ni—Ti alloy.

In a preferred embodiment, the core members 202, 222 are configured to be disposed proximate, more preferably, surround a renal artery, such as illustrated in FIG. 6, wherein core member 222 is shown partially surrounding a renal artery 302, or a structure associated therewith, such as the superior segmental renal artery, inferior segmental renal artery, posterior segmental artery, and inferoanterior segmental renal artery.

In a preferred embodiment, each of the core members 202, 222 are adapted to be configured in at least a circular shape that corresponds to at least one outer surface region of the renal nerve plexus 306 and, hence, renal artery 302 (sec FIGS. 1B and 1C), i.e., a circular shape that surrounds at least 45°, more preferably, at least 180° of the renal nerve plexus 306 and, hence, renal artery 302, as illustrated in FIG. 6.

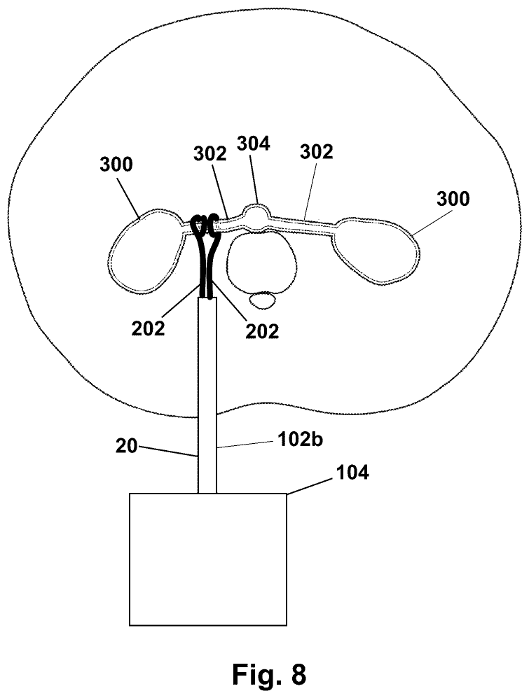

As indicated above, in a preferred embodiment, the catheter systems 100 comprise at least two (2) of the catheter apparatus of the invention, i.e., catheter apparatus 200, 220. In such embodiments, the core members 202, 222 are preferably disposed in opposing directions, wherein a first core member (202 or 222) is positioned whereby the first core member bends or curls in a first direction and the second core member bends or curls in a second direction, wherein the first and second core members preferably surround at least 180°, more preferably, approximately 360° of the renal nerve plexus 306 and, hence, renal artery 302, such as illustrated in FIGS. 5D and 8.

According to the invention, the noted configurations facilitate optimal placement of the core members 202, 222 proximate the renal nerve plexus 306 and, hence, renal nerves 312.

To facilitate the noted configurations and capability, in a preferred embodiment, the core members 202, 222 comprise a shape-memory Ni—Ti alloy (referred to hereinafter as Nitinol®).

It is, however, understood that, although the following exemplary embodiments of the core members of the invention are also described and illustrated herein in connection with core members comprising shape-memory Ni—Ti alloys; specifically, Nitinol®, the invention is not limited to core members comprising Ni—Ti alloys. Indeed, the teachings disclosed herein can also be readily employed to provide core members of the invention comprising other shape-memory alloys, such as, by way of example, Fe—Mn—Si, Cu—Zn—Al, and Cu—Al—Ni alloys.

In some embodiments, the core members 202, 222 are thus adapted to transition from a pre-deployment configuration, wherein the core members 202, 222 can be disposed in a delivery catheter, such as delivery catheter 20, and transition to a post-deployment configuration, i.e., a configuration that facilitates surrounding a renal artery 302, when subjected to a pre-defined critical temperature, i.e., martensite-austinite transition temperature (Ar).

In some embodiments, the pre-defined temperature Ar comprises a temperature in the range of approximately 35.0° C. to approximately 45.0° C. In some embodiments, the pre-defined temperature Ar comprises the temperature of a human body, i.e., approximately 37.0° C.

According to the invention, the critical pre-defined temperature Ar can be reached or provided in two (2) ways: (i) via body temperature, as indicated above, and (ii) via delivery of energy to the core members 202, 222.

As indicated above, the energy delivered to the core members 202, 222 via the energy module 108a can comprise, without limitation, radiofrequency (RF) energy, electrical energy, laser energy, ultrasonic energy, and high-intensity focused ultrasound (HIFU) energy.

In a preferred embodiment, the energy delivered to the core members 202, 222 via the energy module 108a comprises electrical energy. Preferably, the heat generated by the electrical energy is transferred to the entire core members 202, 222, wherein the core members 202, 222 transition to a post-deployment configuration, i.e., a configuration that facilitates surrounding and contacting the renal artery 302 and, hence, renal nerves 312 and/or circumferential tissue 315 thereof.

In some embodiments, the energy provided to the core members 202, 222 is only directed to the distal end region of the core members 202, 222, wherein the distal end region of the core members 202, 222 can similarly transition to a post-deployment configuration that facilitates surrounding the renal artery 302 and, hence, renal nerves 312 and/or circumferential tissue 315 thereof.

The noted energy delivery and resulting configurations of the core members 202, 222 facilitates optimum trackability of the core members 202, 222 (i.e., case of maneuvering around blood vessels and associated blood vessel structures), good flexibility, a low profile, good conformability, and high radial force when deployed.

According to the invention, the core members 202, 222, can also be configured to partially or fully surround the renal artery 302 via a mechanical bending force applied by the operator's manipulation and, thereafter, if necessary or desired, further configured to fully surround the renal artery 302 and, hence, renal nerves 312 via delivery of the electrical energy.

In some embodiments, the core members 202, 222 are adapted and configured to transition to different post-deployment coiled configurations based on the energy delivered and, hence, resulting temperature of the core members 202, 222.

By way of example, in some embodiments, the core members 202, 222 are adapted to transition to a first post-deployment coiled configuration at a first temperature (t1) and thereafter transition to a second post-deployment coiled configuration at a second temperature (t2).

According to the invention, the core members 202, 222 can comprise a two-way or three-way (or higher order, i.e., four-way, five-way, etc.) shape-memory alloy. In some embodiments, the core members 202, 222 comprise a three-way shape-memory alloy, wherein the core members 202, 222 are capable of transitioning from a pre-deployment configuration to first, second and third post-deployment configurations at first, second and third temperatures (t1, t2, t3) above a pre-defined Ar temperature, respectively.

In some embodiments, a cooling agent is perfused into the core members 202, 222 to modulate the temperature of the core members 202, 222 during delivery of the electrical energy thereto, e.g., maintain the temperature below a predetermined temperature, e.g., A_{f}.

As indicated above, in addition to providing energy to transform and configure the core members 202, 222, in some embodiments, the energy delivery module 108a is further adapted to generate and transmit energy proximate the renal nerve plexus 306 to abate activity of, i.e., denervate, the renal nerves 312.

In some embodiments, the energy delivered to the core members 202, 222 thus comprises thermal RF energy using Quantum Molecular Resonance (QMR). According to the invention, the frequency of the RF energy can be greater than or equal to approximately 4.0 MHz, wherein at least a portion of the renal nerve plexus 306 of the renal artery 302 is neutralized.

As indicated above, in a preferred embodiment, the agent delivery module 108b of the renal denervation system 100 is adapted and configured to modulate the delivery of a pharmacological agent, such as a neurolytic agent proximate to the renal nerve plexus 306 and, hence, renal nerves 312 via a catheter assembly, e.g., catheter assemblies 102a, 102b.

In some embodiments, the agent delivery module 108b is further adapted and configured to modulate the delivery of a cooling agent to the core members 202, 222.

According to the invention, the agent delivery module 108b facilitates the delivery of any suitable volume of a pharmacological agent at any suitable rate proximate the renal nerve plexus 306 via a catheter assembly, e.g., catheter assemblies 102a, 102b.

In some embodiments, the agent delivery module 108b is configured to deliver a volume or dose of a pharmacological agent into and though the catheter apparatus 200, 220 and, thereby, proximate the renal nerve plexus 306 in the range of approximately 0.01 cc to approximately 100.0 cc, more preferably, in the range of approximately 0.1 cc to approximately 50.0 cc, when the catheter apparatus 200, 220 are disposed proximate to the renal nerve plexus 306.

In some embodiments, the agent delivery module 108b is adapted and configured to deliver a pharmacological agent at a rate in the range of approximately 0.01 cc/minute to approximately 200.0 cc/minute, more preferably, at a rate in the range of approximately 0.1 cc/minute to approximately 100.0 cc/minute.

According to the invention, the agent delivery module 108b is also adapted and configured to deliver multiple doses of a pharmacological agent.

In a preferred embodiment, the pharmacological agent comprises a neurolytic agent including, but not limited, to ethanol, phenol, glycerol, local anesthetics in relatively high concentration (e.g., lidocaine, bupivacaine, tetracaine, benzocaine, etc.); neurotoxic anti-arrhythmic drugs (e.g., amiodarone and flecainide), botulinum toxin (Botox®), digoxin, cardiac glycosides, guanethidine; heated fluids including heated saline, hypertonic saline, hypotonic fluid, and potassium chloride; and cooled fluids, such as liquid nitrogen.

In some embodiments, the pharmacological agent comprises one of the aforementioned anti-inflammatory agents.

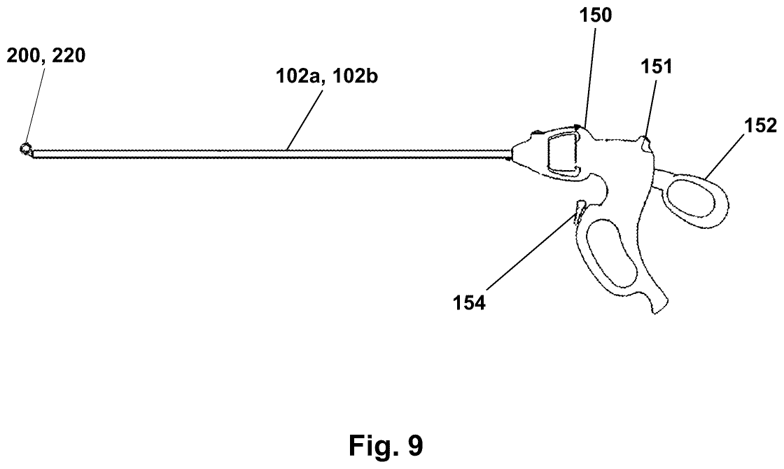

Referring now to FIG. 9, in some embodiments, the renal denervation systems of the invention comprise a hand grip actuator 150, which is in operational communication with the catheter control system 104 via control system connection means 151.

As illustrated in FIG. 9, the hand grip actuator 150 is configured and adapted to guide the delivery catheter 20 and, hence, catheter apparatus 200, 220 proximate the target renal nerve plexus 306 and, hence, renal artery 302.

As illustrated in FIG. 9, in a preferred embodiment, the hand grip actuator 150 comprises first actuation means 152 that is adapted to control the deployment and retraction of the catheter apparatus 200, 220 out of the delivery catheter 20 and positioning thereof proximate the renal artery 302, and second actuation means 154 that is adapted to further modulate the delivery of pharmacological and cooling agents into and through the core members 202, 222 of the catheter apparatus 200, 220 and, thereby, proximate the renal nerve plexus 306.

### Methods of Denervation

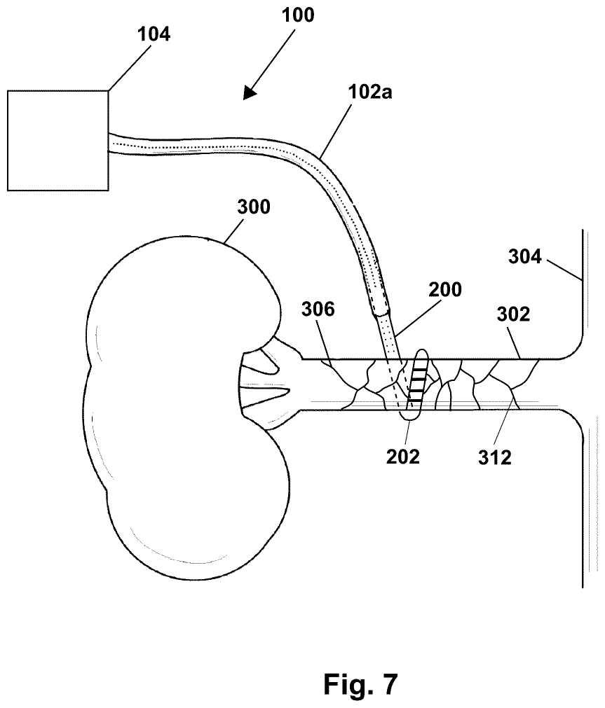

As set forth below, in one preferred embodiment, the method for conducting a renal denervation with a renal denervation system of the invention comprises the following steps:
(i) provide a renal denervation system of the invention, in this instance, a renal denervation system 100 comprising a catheter assembly, such as catheter assembly 102a or 102b;
(ii) initiate the catheter control system 104 of the system 100;
(iii) place the patient in a prone position on an operating table;
(iv) anesthetize the patient's incision site;
(v) create an incision on either the left or right side of the patient's spine at the desired incision site to provide access to the patient's abdominal cavity;
(vi) guide the delivery catheter 20 into and through the incision;
(vii) position the distal end 23b of the delivery catheter 20 proximate to a left or right renal artery 302 of the patient, as illustrated in FIG. 7;
(viii) slidably translating the catheter apparatus 200 into, through and out of the delivery catheter 20;
(ix) transmit first electrical energy to the core member(s) 202 of the catheter apparatus 200, wherein the core member(s) 202 transform to the desired configuration and, thereby, surround and contact the target left or right renal artery 302 of the patient, as also illustrated in FIG. 7;
(x) deliver a pre-determined volume of a neurolytic agent into and through the core member(s) 202, wherein the neurolytic agent is dispersed and out of the fenestrations 206 and delivered proximate renal artery 302 and, thereby, renal nerve;
(xi) retract the catheter apparatus 200 back into the catheter sheath 20; and
(xii) withdraw the catheter assembly from the patient's incision site.

In some embodiments of the invention, after the first electrical energy is delivered to the core member(s) 202, the noted method further comprises the step of delivering second electrical energy the core member(s) 202 to denervate (or further denervate) the renal nerves 312.

In some embodiments, the noted method further comprises the step of delivering a pre-determined volume of a cooling agent into and through the core member(s) 202 to transition the core member(s) 202 from the post-deployment coiled configuration back to the pre-deployment configuration for retraction back into the catheter sheath 20.

According to the invention, the method for conducting renal denervation can be conducted under the guidance of any suitable medical imaging method. In some embodiments, the method for conducting renal denervation is conducted under fluoroscopic (i.e., X-ray) guidance.

In some embodiments, the method for conducting renal denervation is conducted under ultrasound guidance.

In some embodiments, the method for conducting renal denervation is conducted under endoscopic guidance.

In a preferred embodiment, the method for conducting renal denervation is conducted under computed tomography (CT) guidance.

According to the invention, the incision site can comprise any suitable position on a patient's body that provides access to a left or right renal artery 302. By way of example, if a patient is afflicted with a congenitally malrotated kidney, the incision site can be located at a region of a patient's body that provides an operator with access to the renal arteries and/or associated renal artery structures of the malrotated kidney.

In a preferred embodiment, the incision site is located in a dorsal region of a patient's body.

According to the invention, after providing an incision, the delivery catheter 20 and, hence, catheter apparatus 200 can be guided into and through the incision and proximate to the renal artery 302 via the hand grip actuator 150 described herein.

According to the invention, the delivery catheter 20 can also be guided into and through the incision and proximate to the renal artery 302 via other conventional surgical instruments, such as a conventional trocar device.

The delivery catheter 20 can also be guided into and through the incision and proximate to the renal artery 302 using an over-the-wire approach, i.e., using a guidewire.

Referring back to FIG. 1A, in some embodiments, after guiding the delivery catheter 20 into and through the incision, the distal end 23b of the delivery catheter 20 is positioned proximate to the proximal end of the renal artery 302 from the abdominal aorta 304 down to the segmented distal end of the renal artery 302 and associated renal artery structures 320, e.g., segmental renal artery structures.

In some embodiments, the approach angle of the delivery catheter 20 is from below a patient's diaphragm. In some embodiments, a trans-diaphragmatic approach angle is employed when a kidney 300 is positioned in a relatively elevated position in a patient's abdomen.

In some embodiments, after a pre-determined volume of a neurolytic agent is delivered proximate the renal nerve, a second pre-determined volume of a neurolytic agent is delivered thereto, i.e., step (x) is repeated, if necessary or desired. According to the invention, an operator can deliver as many doses of a neurolytic agent as deemed necessary by the operator.

According to the invention, after a pre-determined volume of a neurolytic agent is delivered proximate the renal nerve, a further pharmacological agent, such an aforementioned anti-inflammatory agent, can be delivered proximate the renal nerve.

According to the invention, the methods for conducting renal denervation of the invention can be done laparoscopically by insufflating the abdominal cavity of the patient with carbon dioxide (CO₂) gas.

As will readily be appreciated by one having ordinary skill in the art, the renal denervation apparatus, systems and methods of the invention will provide numerous advantages compared to conventional renal denervation apparatus, systems and methods. Among the advantages are the following:
The provision of renal denervation apparatus, systems and methods, which abate sympathetic renal nerve activity without damaging the renal artery and its associated structures;
The provision of renal denervation apparatus, systems and methods, which abate sympathetic renal nerve activity percutaneously from outside of the renal artery lumen, i.e., extravascularly;
The provision of renal denervation apparatus, systems and methods, which provide precise, consistent, and homogenous distribution of a neurolytic agent proximate to the renal nerve plexus that surrounds the renal artery;
The provision of renal denervation apparatus, systems and methods, which are adapted to deliver precise and controlled (i) energy, e.g., electrical and radiofrequency (RF), heat to catheter members to transform the shape of the members and abate sympathetic renal nerve activity, and (ii) pharmacological agents; particularly, neurolytic agents, proximate the renal nerve plexus to abate sympathetic renal nerve activity;
The provision of renal denervation apparatus and systems, which are adapted to target multiple renal arteries and associated renal artery structures, such as segmental renal arteries, via a single extravascular, percutaneous approach; and
The provision of renal denervation methods, which do not require general anesthesia and can be performed with local anesthesia or moderate conscious sedation of a patient.

What is claimed is:

1. A denervation system for percutaneous abatement of renal nerve activity, comprising:
a delivery catheter, a first catheter apparatus, a second catheter apparatus and a control system, said control system in communication with said first and second catheter apparatus,
said delivery catheter comprising a first internal lumen, said first internal lumen of said delivery catheter configured to receive said first and second catheter apparatus therein,
said first catheter apparatus comprising a first core member, said first core member comprising a first proximal end region, a first distal end region and a first outer surface region,
said first core member further comprising a second internal lumen and a first plurality of fenestrations,
said second internal lumen of said first core member extending from said first distal end of said first core member to said first proximal end of said first core member, said second lumen adapted to receive a pharmacological agent therein,
said first plurality of fenestrations disposed proximate said first distal end region of said first core member and in communication with said second internal lumen, said first plurality of fenestrations sized and adapted to allow said pharmacological agent, when said received in said second internal lumen of said first core member, to be dispersed out of said second internal lumen and delivered proximate a renal artery when said first core member is disposed proximate said renal artery,
said first core member comprising a superelastic shape-memory alloy, whereby said first core member is adapted to transition from a first pre-deployment configuration to a first deployed configuration when said first core member is subjected to a first pre-defined critical temperature,
said first deployed configuration of said core member comprising a first catheter configuration, said first catheter configuration conforming to a first extravascular surface region of said renal artery,
said second catheter apparatus comprising a second core member, said second core member comprising a second proximal end region, a second distal end region and a second outer surface region,
said second core member further comprising a third internal lumen and a second plurality of fenestrations,
said third internal lumen of said second core member extending from said second distal end of said second core member to said second proximal end of said second core member, said third internal lumen adapted to receive said pharmacological agent therein,
said second plurality of fenestrations disposed proximate said second distal end region of said second core member and in communication with said third internal lumen, said second plurality of fenestrations sized and adapted to allow said pharmacological agent, when said received in said third internal lumen of said second core member, to be dispersed out of said third internal lumen and delivered proximate said renal artery when said second core member is disposed proximate said renal artery,
said second core member comprising said superelastic shape-memory alloy, whereby said second core member is adapted to transition from a second pre-deployment configuration to a second deployed configuration when said second core member is subjected to a second pre-defined critical temperature,
said second deployed configuration of said second core member comprising a second catheter configuration, said second catheter configuration conforming to a second extravascular surface region of said renal artery,
said first core member and said second core member further adapted to receive and transmit at least first energy therethrough,
said control system comprising an energy delivery module, an agent delivery module and power supply means,
said energy delivery module adapted to generate and transmit said at least first energy to said first and second core members,
said agent delivery module adapted to modulate delivery of said pharmacological agent to said first and second core members,
said power supply means adapted to provide power to said control system.

2. The denervation system of claim 1, wherein said first catheter configuration of said first core member is in an opposite direction relative to said second catheter configuration of said second core member.

3. The denervation system of claim 1, wherein said superelastic shape-memory alloy comprises a nickel-titanium (NiTi) alloy.

4. The denervation system of claim 1, wherein said at least first energy delivered to and transmitted through said first and second core members comprises electrical energy.

5. The denervation system of claim 4, wherein said electrical energy provides said first and second pre-defined critical temperatures.

6. The denervation system of claim 1, wherein said first core member and said second core member are further adapted to receive and transmit at least second energy therethrough and said energy delivery module is further adapted to generate and transmit said at least second energy to said first and second core members.

7. The denervation system of claim 6, wherein said at least second energy comprises radiofrequency (RF) energy.

8. The denervation system of claim 7, wherein said RF energy abates renal nerve activity.

9. The denervation system of claim 1, wherein said first core member further comprises a first outer sheath disposed proximate said first outer surface region of said first core member.

10. The denervation system of claim 1, wherein said second core member further comprises a second outer sheath disposed proximate said second outer surface region of said second core member.

11. The denervation system of claim 1, wherein said pharmacological agent is adapted to induce neutralization of a renal nerve.

12. The denervation system of claim 11, wherein said pharmacological agent comprises a neurolytic agent selected from the group consisting of ethanol, phenol, glycerol, lidocaine, bupivacaine, tetracaine, benzocaine, amiodarone, flecainide, botulinum toxin (Botox®), digoxin, a cardiac glycoside, guanethidine, heated saline, heated hypertonic saline, heated hypotonic fluid, heated potassium chloride and liquid nitrogen.

13. The denervation system of claim 1, wherein said pharmacological agent comprises an anti-inflammatory agent selected from the group consisting of methylprednisolone, triamcinolone, betamethasone and dexamethasone.

14. The denervation system of claim 1, wherein said pharmacological agent comprises an antibiotic selected from the group consisting of an aminoglycoside, a cephalosporin, chloramphenicol, clindamycin, an erythromycin, a fluoroquinolone, a macrolide, an azolide, metronidazole, a penicillin, a tetracycline, trimethoprim-sulfamethoxazole, gentamicin and vancomycin.
